(19) United States
(12) Patent Application Publication
(10) Pub. No.: US 2006/0161048 A1
(43) Pub. Date: Jul. 20, 2006

(54) Flexible video scope extension and methods

(76) Inventor: John B. Squicciarini, Aliso Viejo, CA (US)

Correspondence Address: FISH & RICHARDSON, PC, P.O. BOX 1022, MINNEAPOLIS, MN, 55440-1022, US

(21) Appl. No.: 11/225,381

(22) Filed: Sep. 12, 2005

Related U.S. Application Data
(60) Provisional application No. 60/608,810, filed on Sep. 10, 2004.

(51) Int. Cl. A61B 1/06; A61B 1/04

(52) U.S. Cl. 600/160; 600/179; 600/170; 600/109; 600/130; 600/131; 600/127

(57) ABSTRACT

An endoscopic device includes a hand-held base, a camera connected with the hand-held base, and a light source connected with the hand-held base for illuminating an area above the camera. The device further includes a lens structure coupled to the hand-held base and positioned proximate the camera and the light source. A removable, disposable enclosure of the lens structure includes a cylindrical body formed of a rigid, transparent material, a proximal end for being removably coupled to the hand-held base, and a distal end that terminates in a rounded transparent tip.

### CROSS REFERENCE TO RELATED APPLICATIONS

The present application claims priority under 35 U.S.C. §119 to U.S. Provisional Application Ser. No. 60/608,810, filed Sep. 10, 2004, entitled FLEXIBLE VIDEO SCOPE EXTENSION AND METHODS, the disclosure of which is incorporated herein by reference.

### BACKGROUND

There are numerous flexible endoscopic devices known in the art, and the specific use will at least in part determined the specific configuration of the endoscope. However, and regardless of the particular use of the endoscope, heretofore known devices will typically fall within one of two general categories.

In one category, the light source and/or the camera is coupled to the flexible endoscope at the handle or controller that is located outside of the person being examined. In the other category, the light source and/or the camera is coupled to the flexible endoscope at the terminal portion that is advanced into the patient. Depending on the particular use, the body of the endoscopic device may be rigid or flexible, and movement of the flexible portion is typically effected via a hand-held controller. Thus, the light source and/or the camera are either on a distal and/or on a proximal end. Consequently, configuration flexibility is typically not achieved with known endoscopes, and a change in a procedure will often necessitate a change of endoscopic device during the procedure. Therefore, while there are numerous endoscopic devices and methods known in the art, all or almost all of them suffer from one or more disadvantages. Thus, there is a need for an improved endoscopic device.

### SUMMARY

This document describes an endoscopic device for penetrating, illuminating and taking video images of a human cavity.

In one aspect, an endoscopic device includes a hand-held base, a camera connected with the hand-held base, and a light source connected with the hand-held base for illuminating an area above the camera. The device further includes a lens structure coupled to the hand-held base and positioned proximate the camera and the light source. A removable, disposable enclosure of the lens structure includes a cylindrical body formed of a rigid, transparent material, a proximal end for being removably coupled to the hand-held base, and a distal end that terminates in a rounded transparent tip.

The details of one or more embodiments are set forth in the accompanying drawings and the description below. Other features and advantages will be apparent from the description and drawings, and from the claims.

### BRIEF DESCRIPTION OF THE DRAWINGS

These and other aspects will now be described in detail with reference to the following drawings.

FIG. 1 shows an endoscopic device in accordance with an exemplary embodiment.

FIG. 2 shows an endoscopic device having a hand-held base and a lens structure in accordance with an exemplary embodiment.

FIG. 3 depicts exemplary tips and enclosures thereof.

FIG. 4 depicts coupling of the tip enclosures to an endoscopic device.

FIG. 5 illustrates a power supply and other controls of a device.

FIG. 6 illustrates various tip enclosures.

FIG. 7 depicts an integrated tip enclosure, light source and camera.

FIG. 8 illustrates a tip enclosure extension.

FIG. 9 shows a tip enclosure and flexible optic fiber extension.

FIG. 10 shows a device with a tip enclosure having a flexible fiber optic extension.

FIGS. 11 and 12 illustrate various angles of view in accordance with exemplary embodiments.

FIGS. 13 and 14 shows a tip enclosure in accordance with an alternative embodiment.

FIGS. 15-17 illustrate a dilator mechanism and uses thereof in combination with an endoscopic device.

Like reference symbols in the various drawings indicate like elements.

### DETAILED DESCRIPTION

This document describes an endoscopic device that can be configured such that a tip is removably coupled to a hand-held base. A fiber optic or other light guide portion in the tip receives light from a light source in the hand-held base. A camera, such as a video chip, in the hand-held base receives image information (e.g., reflected and/or emitted light from the tissue to be examined) via a fiber optic or other light guide portion in the tip. In some embodiments, the distal end of the tip includes a lens, which may further be detachable from the tip. The tip and/or lens is preferably disposable or formed of a disposable material such as plastic or acrylic.

In an exemplary embodiment, the hand-held base includes the camera, the light source, the image processor, the power supply, and a data interface to relay the image signal to a monitor or other display device. For example, the camera may comprise a charge coupled device (CCD) chip. The CCD chip may have a size of ¼, ⅓, or ⅙ inch with 400,000 total pixels, and scanned at a rate of 60 Hz using 400 lines. Suitable light sources include LED and incandescent light sources. Light filters (optical or electronic) can also be used in order to generate a light source of particular, predetermined light characteristics (i.e. luminescence, brightness, etc.) Preferably, illumination by the light source is at least 1 lux. Image processing to produce an NTSC image can be done using electronic components, and freeze-frame and continuous output can be provided. The power supply is preferably included in the hand-held base and includes a rechargeable battery (Li-ion or otherwise). The hand-held base can further include data interfaces for transmission of the CCD signal, and the video signal from the image processor can be output via a wireless interface (Bluetooth, microwave, infrared, etc.) and/or a wired interface (USB2.0, Firewire, etc.).

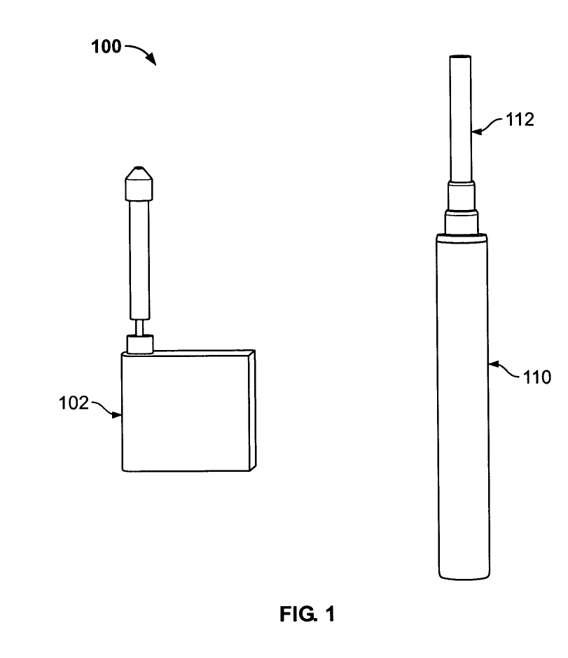

FIG. 1 depicts an endoscopic device 100 according to an exemplary embodiment. The endoscopic device 100 delivers high-resolution, preferably wireless video reproduction and transmission of detailed examinations of body cavities. The device 100 includes a hand-held base 110 and a tip 112. The tip 112 includes a lighting source (not shown) and an image collection mechanism (not shown), preferably a video camera. The device 100 can be combined with a video receiver 102, such as a microwave video receiver, to receive video signals from the device 100. The video receiver 102 can be connected to a monitor, a television, a computer, a tape recorder or other digital video recorder.

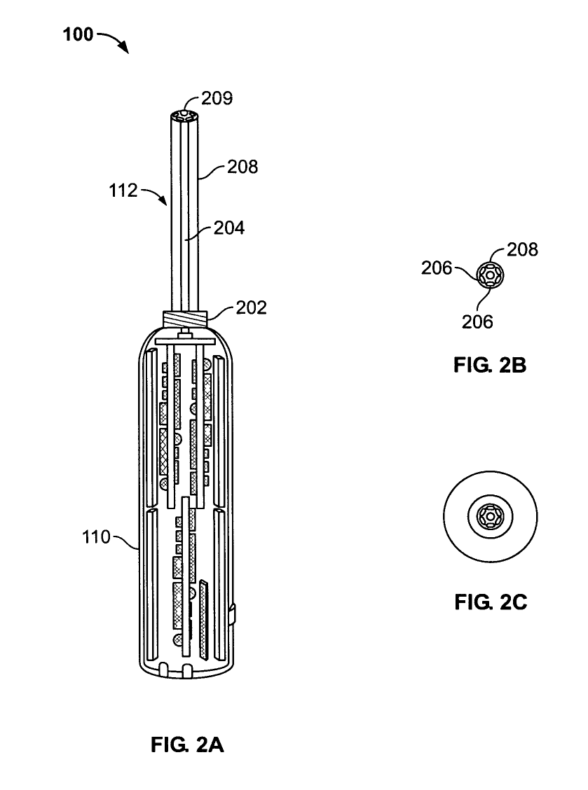

FIG. 2 depicts various views of an endoscopic device 100 in which a camera 202, a light source 206, and other electronic components are disposed in the hand-held base 110 and projected through a lens structure 204 disposed within a lens enclosure 208. The lens structure 204 can magnify image signals being received by the camera 202, and/or control light from the light source 206 to illuminate tissue being examined. Alternative, the camera 202 and light source 206 can be disposed within the lens enclosure 208, and controlled by circuitry in the hand-held base 110.

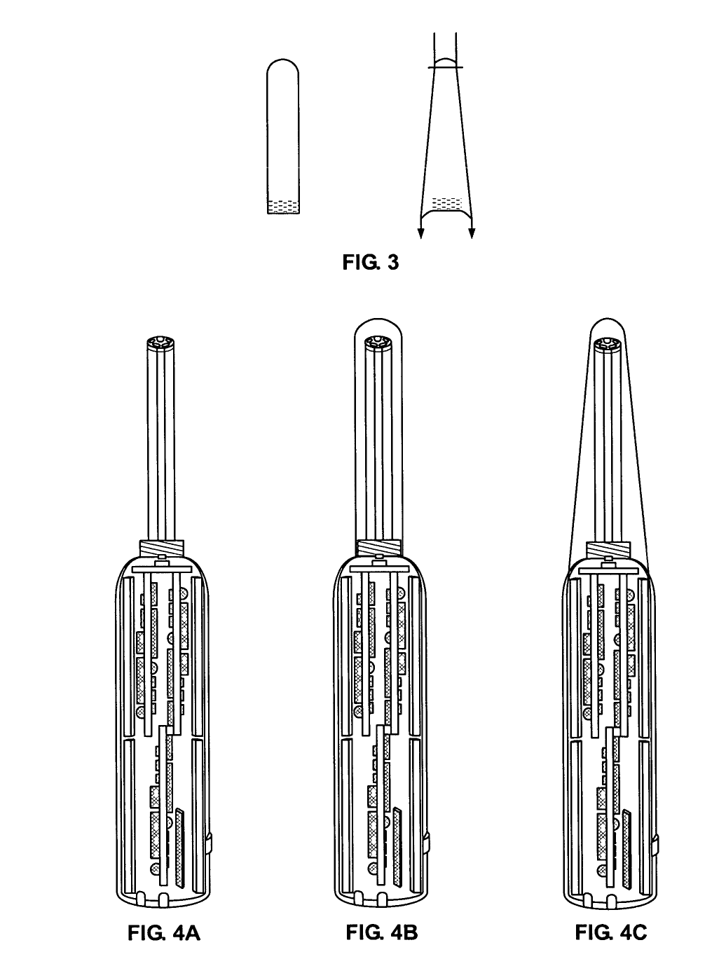

FIG. 3 depicts exemplary tips and enclosures thereof. The inner diameter of the tip enclosure can be just large enough to fit over a lens extension of an endoscopic device. The tip enclosure can be formed of plastic, Teflon-coated plastic, glass, or Teflon-coated acrylic. The tip enclosures also serve to spread open a cavity of the patient being examined, to allow full video capture and illumination without being blocked by body membranes or other tissue.

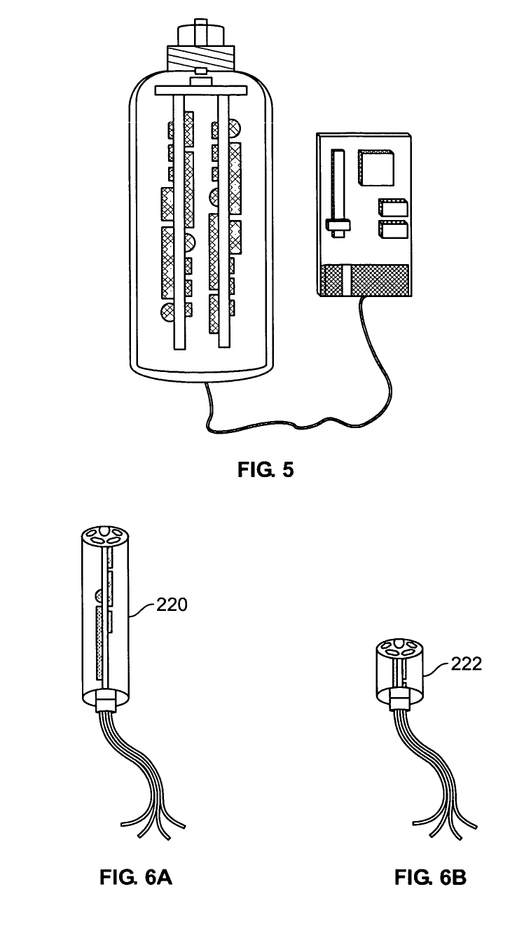

FIG. 4 depicts coupling of the tip enclosures shown in FIG. 3 to the device of FIG. 2. The power supply and other controls may alternatively be disposed outside of the hand-held base, as shown in FIG. 5. The controls include an on/off switch, a camera control, an illumination control (including illumination intensity control), RGB color control, lens adjustment, and white balance adjustment. Other controls can include a wireless antenna and circuit for wireless communication of video data from the device, as well as other interface connections, such as USB, Firewire, analog audio and/or video, and other interfaces or controls.

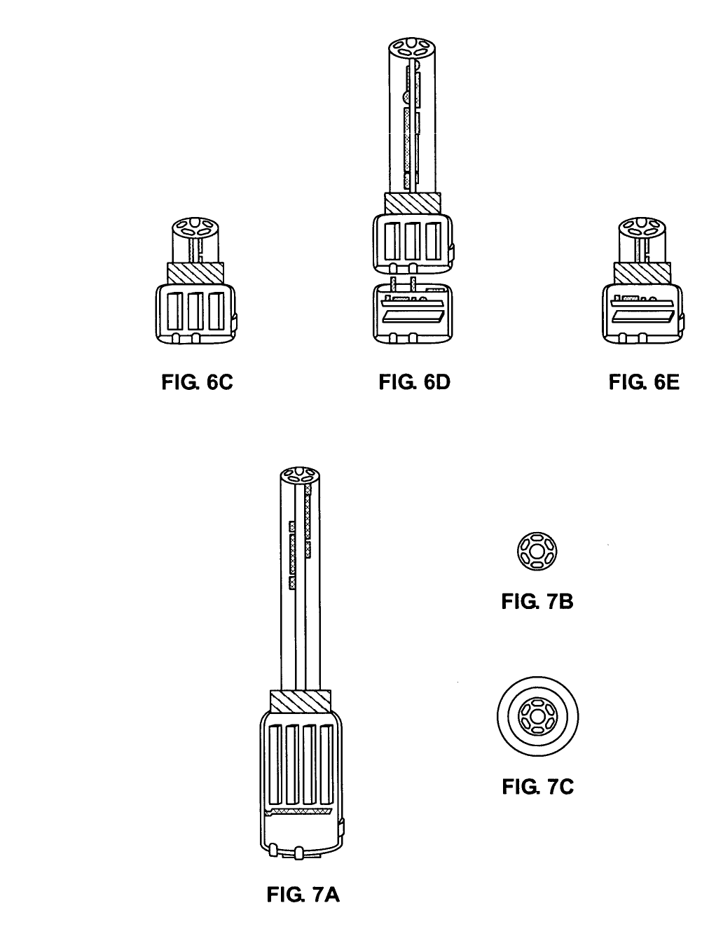

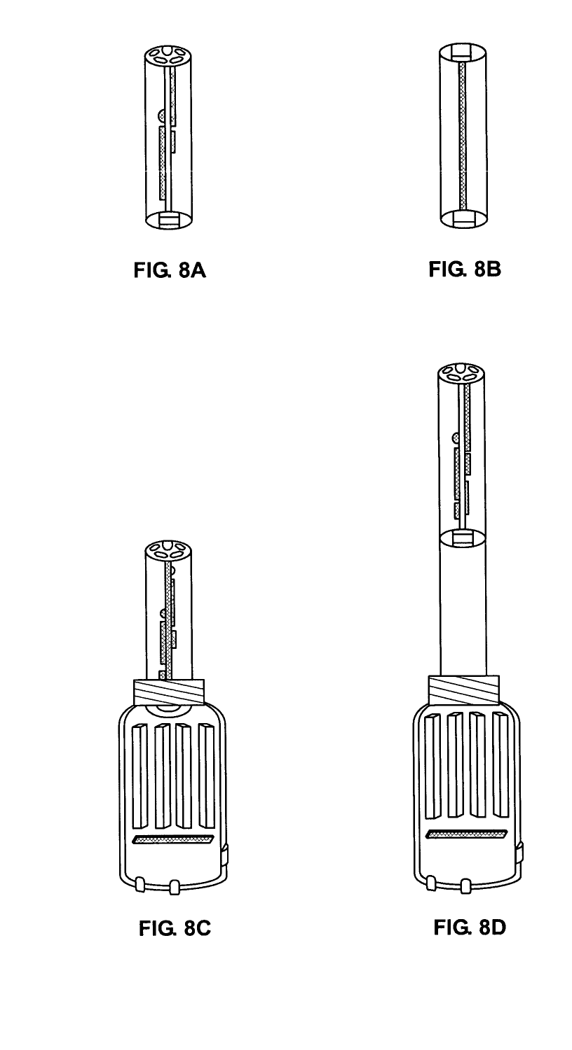

Exemplary tip configurations are depicted in FIG. 6. The tip can include a tall extended lens 220 or short compressed lens 222. The tip may also include an internal battery controller with wireless transmitter. The tip configurations that include the camera and/or the LED in the tip are depicted in FIG. 7. An optional tip extender, having an extension shaft that extends to tip from a compressed mode to an extended mode, is shown in FIG. 8.

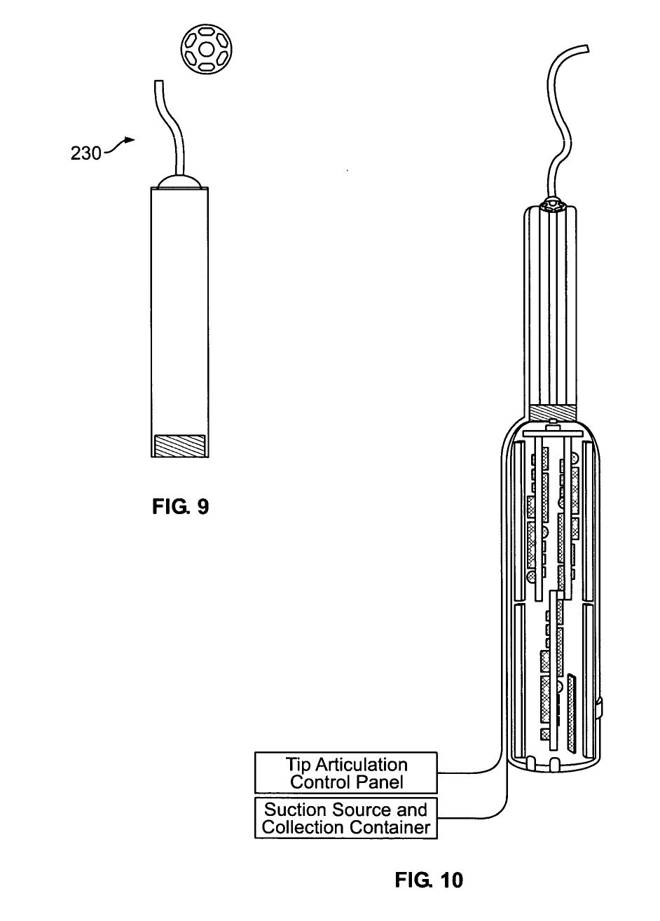

As shown in FIG. 9, the tip may include a flexible fiber optic extension 230 through which illumination and/or the image is transferred via the tip to the camera in the hand-held base. The length of the fiber optic extension 230 is based on an application, and can range from one-half to twelve inches or more. The fiber optic extension 230 is illuminated by a lighting source on the tip of the camera, and fits snugly against the camera and light source so that no video return signal is lost or misdirected, and that all of the desired video is directed to the fiber optic extension 230. An exemplary device using such flexible tip is depicted in FIG. 10. Preferably, the flexible tip is actuated using an actuator that is external to the tip (and hand-held base), and in even more preferred aspects, the tip may further include a working tool (e.g., suction line or fluid line).

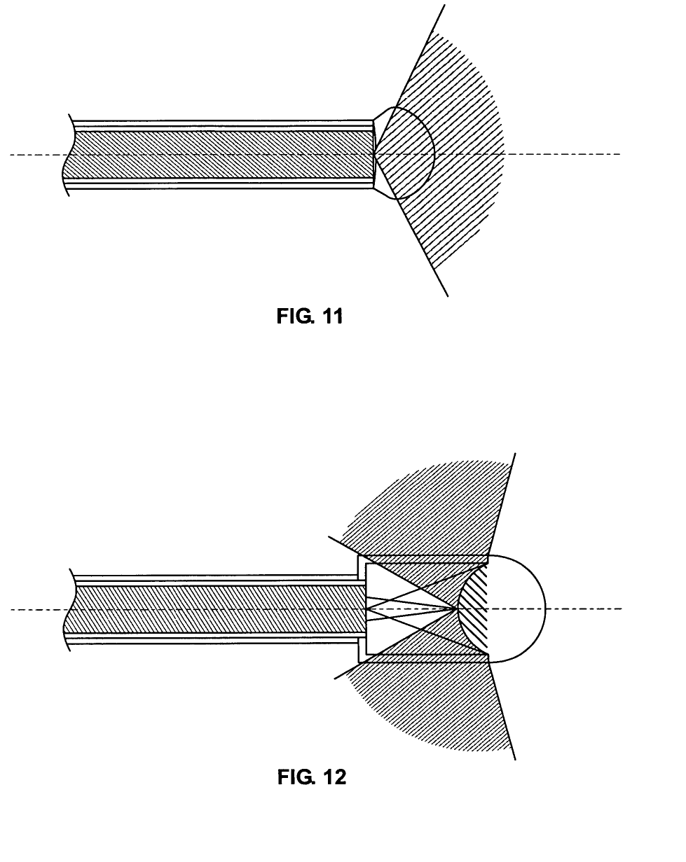

It is also contemplated that the distal tip may be configured to provide a forward viewing tip and/or a omni-directional viewing tip. Exemplary forward viewing tips and omni-directional viewing tips are depicted in FIGS. 11 and 12, respectively. Here, the forward viewing tip has a front element with dual purpose: the front element provides a smooth spherical surface that acts as a dilator and spreads the surrounding tissue reducing the patient discomfort, and acts as an optical element in front of the imaging optics to increase the overall magnification. The front element can be configured as a part of the imaging system to provide a microscope type of magnification and resolution at the near focus, while the rear surface of the element is an aspheric surface designed to eliminate the back reflections from the LEDs into the field of view.

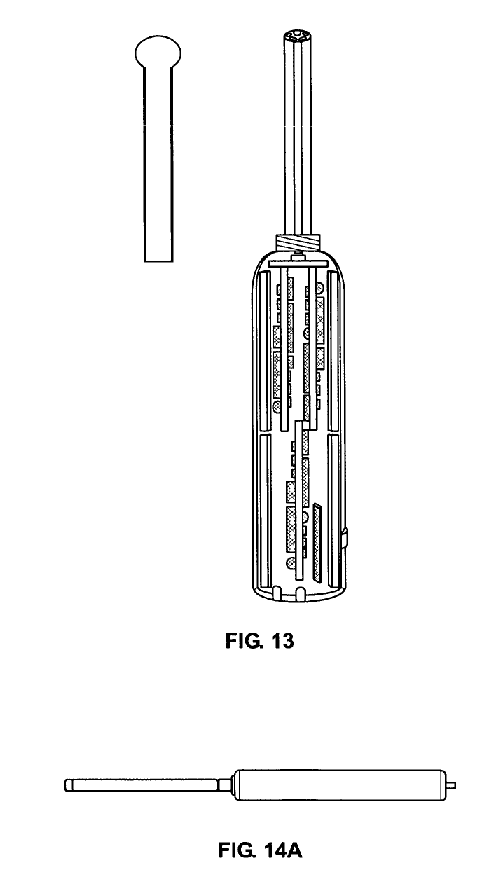

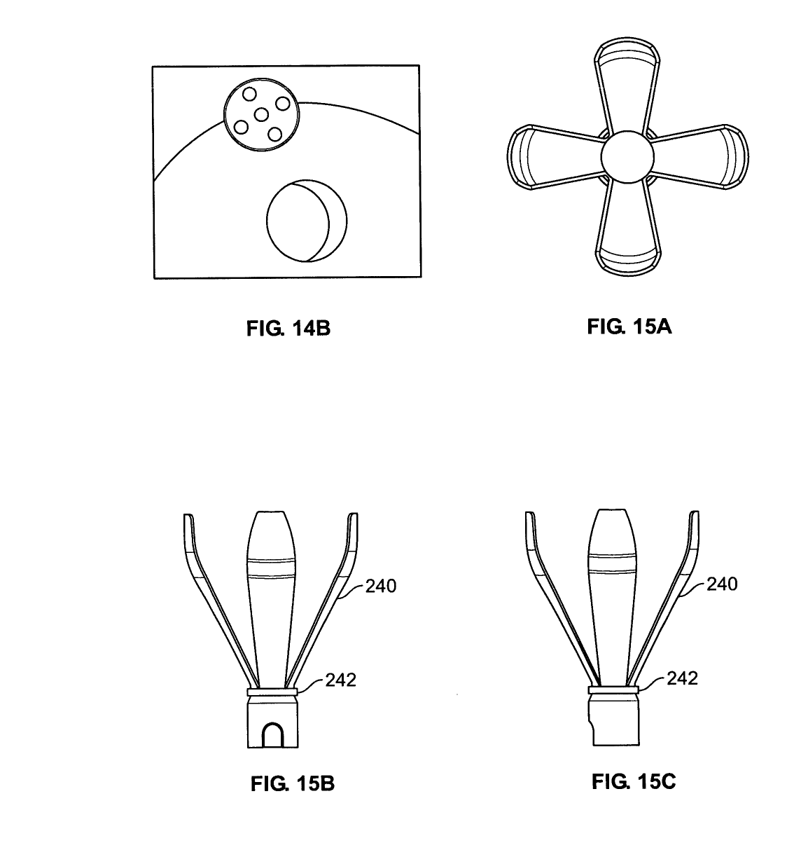

Exemplary aspects of such tips are depicted in FIGS. 13 and 14. The omni-directional viewing tip has an outer tube with a clear acrylic cylinder and a spherical front element to provide an omni-directional view of the surrounding walls or tissue. Here, the front element is part of the imaging system to provide a 360 degrees peripheral field of view. The front surface of the element is spherical and acts as a tissue dilator, while the rear surface of the element can be designed as the one or several aspheric refractive or reflective surfaces to provide a distortion-free imaging of the surrounding tissue. The resulting image is then in a shape of a donut and can be viewed on a monitor or display screen. However, such an image can also be unfolded into a panoramic continuous flat image using software.

The tips are generally configured such that the tip provides sufficient optical contact between the light guides for the camera and/or light source of the tip and the camera and/or light source that are located in the hand-held base. For example, the tip may be screwed into place using the proximal end of the tip. Alternatively, a bayonet lock or other temporary fastener may be employed to secure the tip to the hand-held base. The tip and the fastening mechanism are fabricated from material that can be sterilized and which allow coupling of the tip to the hand-held base. Accordingly, the base may include a disposable sterile cover. In such configurations, the device can be repeatedly used without undergoing sterilization by providing a disposable sterile cover to the base and a sterile disposable tip. Alternatively, the entire device may be covered by a disposable sterile cover. Further, the tip may be at least partially covered by a sterile and disposable sheath that provides magnification or other optical properties (e.g., modified viewing angle, etc.).

The hand-held base preferably has a maximum length of less than fifteen inches, more typically less than ten inches, and preferably less than eight inches. Similarly, the diameter (or maximum width) of the hand-held base is less than three inches, more typically less than two inches, and preferably ranging from 0.5 to one inch in width. Suitable tips can have a length of between about 0.1 inches to 10 inches, and even longer. However, it is preferred that a length of the tip is between about 0.5 inches to 4 inches. Thus, as viewed in terms of a three-part device, the camera and/or light source of the device is disposed in the central third, while the distal third includes the disposable tip and the proximal third includes the hand-held base.

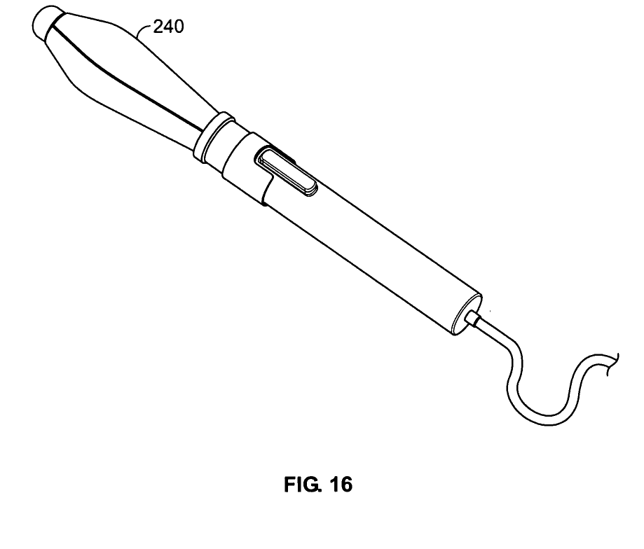

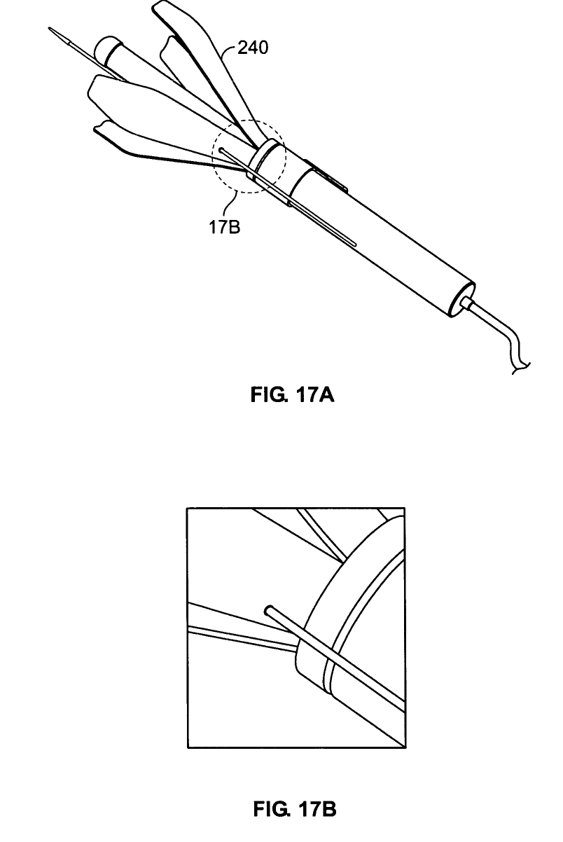

Additionally, or alternatively, contemplated tips may also include a tissue dilator 240. An exemplary dilator 240, which may be integral to the tip or removably attached to the tip, is depicted in FIG. 15. A control ring 242 locks the dilator elements in a closed position, and the dilator elements spread out when the ring is moved towards the base element. FIG. 16 shows the dilator 240 of FIG. 15 coupled to an exemplary device, wherein the dilator 240 is in a closed configuration. Where desired, additional working tools may not only be located in the tip, but also within a space that is defined between the tip and the dilator arms, wherein the working tools are preferably movable as depicted in FIG. 17.

The lens at the distal end of the tip may have various optical properties. For example, the distal end of the tip can be shaped with a curvature that forms part of the lens. In such configurations, the distal end of the tip may have a central section that includes the optical fibers that transmit the reflected to and/or emitted light from the tip to the camera, while a plurality of circumferentially disposed sections may include optical fibers that provide the light from the light source to tissue being examined. In some embodiments, such tips provide a homogeneous illumination to the target area. Where desired, a reflective or filtering coating may be applied to the distal end of the tip to reduce or even eliminate direct light transmission from the light source optical fibers to the camera optical fibers.

Although a few embodiments have been described in detail above, other modifications are possible. Other embodiments may be within the scope of the following claims.

1. An endoscopic device comprising:
a hand-held base;
a lens structure coupled to the hand-held base; and
a tip enclosure that is removably coupled to the hand-held base and sized to enclose the lens structure.

2. A device in accordance with claim 1, wherein the lens structure includes a light source.

3. A device in accordance with claim 2, wherein the hand-held base includes a light guide for receiving light from the light source.

4. A device in accordance with claim 1, wherein the lens structure includes a camera.

5. A device in accordance with claim 4, wherein the hand-held base includes a video chip for receiving video signals from the camera.

6. A device in accordance with claim 4, wherein the tip enclosure further includes a lens.

7. A device in accordance with claim 6, wherein the lens is configured to be placed proximate an aperture of the camera.

8. A device in accordance with claim 1, further comprising a dilator coupled to the hand-held base and around at least a portion of the tip enclosure.

9. A device in accordance with claim 2, wherein the light source includes one or more light emitting diodes (LEDs).

10. A device in accordance with claim 4, wherein the camera includes a charge coupled device (CCD) image receiver.

11. A device in accordance with claim 1, further comprising a wireless transmission device.

12. A device in accordance with claim 1, further comprising a power source disposed within the hand-held base.

13. A device in accordance with claim 1, wherein the tip enclosure is formed of acrylic.

14. A device in accordance with claim 1, wherein the tip enclosure includes an outer coating of a hydrophobic material.

15. A device in accordance with claim 1, wherein the hand-held base includes control devices and circuitry.

16. An endoscopic device comprising:
a hand-held base;
a camera connected with the hand-held base;
a light source connected with the hand-held base, for illuminating an area above the camera; and
a lens structure coupled to the hand-held base and positioned proximate the camera and the light source.

17. A device in accordance with claim 16, further comprising a tip enclosure that is removably coupled to the hand-held base and sized to enclose the lens structure.

18. A device in accordance with claim 17, wherein the tip enclosure is cylindrical, having a proximal end for coupling to the hand-held base and a distal end terminating in a rounded tip.

19. A device in accordance with claim 18, wherein the tip enclosure has a diameter of no greater than two inches.

20. An enclosure for an endoscopic device that includes a hand-held base, a camera, a light source for illuminating an area above the camera, and a lens structure coupled to the hand-held base and positioned proximate the camera and the light source, the enclosure comprising:
a cylindrical body formed of a rigid, transparent material;
a proximal end for being removably coupled to the hand-held base; and
a distal end that terminates in a rounded transparent tip.
